(19) United States
(12) Patent Application Publication
(10) Pub. No.: US 2020/0345274 A1
(43) Pub. Date: Nov. 5, 2020

(54) SYSTEMS AND METHODS FOR MONITORING RESPIRATION AND MOTION

(71) Applicant: ADVANCED TELESENSORS, INC., Austin, TX (US)

(72) Inventors: Sajol Ghoshal, Austin, TX (US); David Kramer, Cedar Park, TX (US); Jaime Martinez, Austin, TX (US)

(21) Appl. No.: 16/862,203

(22) Filed: Apr. 29, 2020

Related U.S. Application Data
(60) Provisional application No. 62/841,033, filed on Apr. 30, 2019.

(51) Int. Cl. A61B 5/113; A61B 5/05; A61B 5/00; A61B 7/00

(52) U.S. Cl. CPC A61B 5/113; A61B 5/05; A61B 5/746; A61B 5/747; A61B 2503/04; A61B 5/7264; A61B 5/0077; A61B 7/003; A61B 2503/06; A61B 5/4818

(57) ABSTRACT

Systems and methods for remotely sensing movement of a mammalian subject utilize a radio frequency (RF) transmitter configured to impinge a RF signal on tissue of a mammalian subject, a RF receiver configured to receive a reflected RF signal, a processor configured to separately identify presence of respiratory and non-respiratory motion of the mammalian subject, and a memory configured to store processes signal values generated by or derived from the processor, wherein the processor is configured to compare one or more processed signals against one or more stored processed signal values, and detect a health state or health condition of the mammalian subject. Sleep apnea and respiratory events may be detected. In one embodiment, motion of a human infant may be mapped over time, and motion trends may be used to assess proper development of the infant.

### CROSS-REFERENCE TO RELATED APPLICATION(S)

This application claims priority to U.S. Provisional Patent Application No. 62/841,033 filed on Apr. 30, 2019, wherein the entire disclosure of the foregoing application is hereby incorporated by reference herein.

### TECHNICAL FIELD

Subject matter herein relates to detection of respiration and motion of a mammal's body.

### BACKGROUND

Mammal beings are living organisms that consume oxygen and expel carbon dioxide through a respiration system that consists of a mouth, trachea, bronchi, and lungs. During the respiration process, air is inhaled through the mouth, trachea, and bronchi into the lungs where the oxygen is exchanged for carbon dioxide via the alveoli. The air is then exhaled through the reverse path. To enable this process, the diaphragm muscles expand and contract to perform the breathing function. Depending upon the mammal, the torso which contains the majority of the respiration organs including the lungs will mechanically expand and contract. The amount of torso displacement can vary from a few mm to cm, depending upon the mammal species, the size of the mammal, and the rate of respiration.

A nominally healthy mammal will normally breathe and undergo motion throughout its life. Movement is defined as physical displacement of the mammal's body utilizing mobility limbs such as arms and legs. There can be occasions where respiration and/or movement classifications can be indicative of anomalies that may need intervention. A lack of respiration and motion may indicate respiration failure (apnea) that has left the mammal unresponsive. A lack of respiration with extreme motion may indicate a choking or suffocation event in which the mammal is struggling to breathe. Recent research has indicated that motion tracking and analysis can be indicative of normal or abnormal newborn infant development (See, e.g., Zuzarte, et al., “Quantifying Movement in Preterm Infants Using Photoplethysmography,” Annals of Biomedical Engineering, Vol. 47, pp. 646-658 (2019).

Need exists for improved systems and methods for detecting health conditions that may be correlated to respiration and motion to address limitations of existing systems and methods in the art.

### SUMMARY

The present disclosure relates in various aspects to devices and methods utilizing reflectometric detection of a mammalian subject (e.g., a human) in order to detect health states or health conditions that may be correlated to respiration and/or motion (e.g., including compliance with or deviation from patterns thereof). Reflected RF signals are processed to separately identify presence of respiratory motion and non-respiratory motion of the mammalian subject. Processed signals may be compared to one or more stored processed signal values (e.g., baseline values) and used to detect at least one health state or health condition of the mammalian subject. By detecting and processing respiratory motion and non-respiratory motion simultaneously, multiple important items may be identified. Motion may be tracked over time to provide a baseline of “normal” movement. Deviations from normal movement or deviations from overall movement progression over time can be indicative of health-related states or conditions, including developmental issues. Detection of respiration rate that is too high can signal hyperventilation. Detection of respiration rate that is too low or in absence (apnea) can indicate a respiration failure that may need immediate medical response. Moreover, detection of respiration rate and/or movement can indicate consciousness and/or activity level of the subject.

In one aspect, the disclosure relates to a system for remotely sensing movement of a mammalian subject. The system comprises: a radio frequency (RF) transmitter configured to transmit a RF signal for impingement on tissue of the mammalian subject; a RF receiver configured to receive a RF signal comprising a reflection of the RF signal impinged on tissue of the mammalian subject; at least one processor configured to process the received RF signal to separately identify presence of respiratory motion of the mammalian subject and presence of non-respiratory motion of the mammalian subject; and a memory configured to store processed signal values generated by or derived from the at least one processor indicative of at least one of (i) respiratory motion of the mammalian subject or (ii) non-respiratory motion of the mammalian subject; wherein the at least one processor is further configured to compare one or more processed signals generated by or derived from the at least one processor against one or more stored processed signal values, and detect at least one health state or health condition of the mammalian subject.

In certain embodiments, the at least one processor is additionally configured to generate an alarm signal responsive to detection of at least one health state or health condition correlated to deviation from baseline conditions for respiratory and/or non-respiratory movement of the mammalian subject.

In certain embodiments, the at least one processor is additionally configured to summon human and/or medical assistance for the mammalian subject responsive to detection of at least one health state or health condition correlated to deviation from baseline conditions for respiratory and/or non-respiratory movement of the mammalian subject.

In certain embodiments, the at least one health state or health condition of the mammalian subject comprises consciousness or lack of consciousness of the mammalian subject.

In certain embodiments, the at least one health state or health condition of the mammalian subject comprises a respiratory trauma event experienced by the mammalian subject, an apnea event experienced by the mammalian subject, a bradypnea event experienced by the mammalian subject, a hyperventilation event experienced by the mammalian subject, and/or a choking or suffocating event experienced by the mammalian subject.

In certain embodiments, the processed signals generated by or derived from the at least one processor comprise one or more baseline values indicative of at least one pattern of normal movement of the mammalian subject, and the detection of the at least one health state or health condition of the mammalian subject comprises deviation from the at least one pattern of normal movement of the mammalian subject.

In certain embodiments, the at least one processor is configured to generate the one or more baseline values using an artificial intelligence engine.

In certain embodiments, the transmitted RF signal comprises one or more of: a microwave frequency signal, a pulsed RF signal, a swept frequency RF signal, or a static RF signal.

In certain embodiments, the system further comprises at least one RF antenna associated with one or more of the RF transmitter or the RF receiver. In certain embodiments, the at least one RF antenna comprises a directional RF antenna. In certain embodiments, at least one of the RF transmitter or the RF receiver comprises a RF antenna array. In certain embodiments, the RF transmitter comprises a first RF antenna array, and the RF receiver comprises a second RF antenna array. In certain embodiments, the RF transmitter comprises a directional RF antenna mounted and aligned to point at a torso of the mammalian subject.

In certain embodiments, the mammalian subject comprises a human infant or child, and the RF transmitter is mounted and aligned to point at a torso of the human infant or child when the human infant or child is present in a medical apparatus, a crib, a bed, or an infant safety seat.

In certain embodiments, the mammalian subject comprises a human infant, and the system is configured to detect motion of the human infant over time, to map motion of the human infant over time to generate a baseline, and to utilize human infant motion trends to determine whether the human infant is undergoing proper development

In certain embodiments, the system further comprises a display configured to display at least one of the following items relating to the mammalian subject: respiration rate, respiration history, alarm state, alarm history, non-respiratory motion history, and baseline values indicative of a pattern of normal movement.

In certain embodiments, the system further comprises a wired or wireless communication device configured to permit communication between a networked device and one or more of the memory or the at least one processor.

In certain embodiments, the at least one processor comprises a plurality of processors.

In certain embodiments, the system further comprises a camera configured to image the mammalian subject, wherein the memory is configured to store one or more still images or videos of the mammalian subject. In certain embodiments, when an alarm signal is detected, the memory is configured to store one or more still images or videos of the mammalian subject in association with the processed signal values generated by or derived from the at least one processor indicative of at least one of (i) respiratory motion of the mammalian subject or (ii) non-respiratory motion of the mammalian subject.

In certain embodiments, the system further comprises a microphone configured to capture sounds generated by the mammalian subject, wherein the memory is configured to store one or more sounds generated by the mammalian subject. In certain embodiments, when an alarm signal is detected, the memory is configured to store one or more sounds generated by the mammalian subject in association with the processed signal values generated by or derived from the at least one processor indicative of at least one of (i) respiratory motion of the mammalian subject or (ii) non-respiratory motion of the mammalian subject.

In another aspect, the disclosure relates to a method for detecting at least one health state or health condition of a mammalian subject. The method comprises: transmitting a radio frequency (RF) signal to impinge on tissue of the mammalian subject; receiving a RF signal comprising a reflection of the RF signal impinged on tissue of the mammalian subject; processing the received RF signal utilizing at least one processor to separately identify presence of respiratory motion of the mammalian subject and presence of non-respiratory motion of the mammalian subject; storing processed signal values generated by or derived from the at least one processor in a memory, the processed signal values being indicative of at least one of (i) respiratory motion of the mammalian subject or (ii) non-respiratory motion of the mammalian subject; and utilizing the at least one processor, comparing one or more processed signals generated by or derived from the at least one processor against one or more stored processed signal values, and responsive to the comparing, detecting at least one health state or health condition of the mammalian subject.

In certain embodiments, the method further comprises automatically generating an alarm signal responsive to detection of at least one health state or health condition correlated to deviation from baseline conditions for respiratory and/or non-respiratory movement of the mammalian subject.

In certain embodiments, the method further comprises automatically summoning human and/or medical assistance for the mammalian subject responsive to detection of at least one health state or health condition correlated to deviation from baseline conditions for respiratory and/or non-respiratory movement of the mammalian subject.

In certain embodiments, the at least one health state or health condition of the mammalian subject comprises consciousness or lack of consciousness of the mammalian subject.

In certain embodiments, the at least one health state or health condition of the mammalian subject comprises a respiratory trauma event experienced by the mammalian subject, an apnea event experienced by the mammalian subject, a bradypnea event experienced by the mammalian subject, a hyperventilation event experienced by the mammalian subject, and/or a choking or suffocating event experienced by the mammalian subject.

In certain embodiments, the processed signals generated by or derived from the at least one processor comprise one or more baseline values indicative of at least one pattern of normal movement of the mammalian subject, and the detection of the at least one health state or health condition of the mammalian subject comprises identification of deviation from the at least one pattern of normal movement of the mammalian subject.

In certain embodiments, the method further comprises generating the one or more baseline values using an artificial intelligence engine.

In certain embodiments, the transmitted RF signal comprises one or more of: a microwave frequency signal, a pulsed RF signal, a swept frequency RF signal, or a static RF signal.

In certain embodiments, at least one of the following items (a) or (b) is performed with at least one RF antenna array: (i) the transmitting of a RF signal to impinge on tissue of the mammalian subject, or (ii) the receiving of a RF signal comprising a reflection of the RF signal impinged on tissue of the mammalian subject.

In certain embodiments, the transmitting of a RF signal to impinge on tissue of the mammalian subject is performed with a first RF antenna array, and the receiving of a RF signal comprising a reflection of the RF signal impinged on tissue of the mammalian subject is performed with a second RF antenna array

In certain embodiments, the mammalian subject comprises a human infant or child, and the RF transmitter is mounted and aligned to point at a torso of the human infant or child when the human infant or child is present in a medical apparatus, a crib, a bed, or an infant safety seat.

In certain embodiments, the mammalian subject comprises a human infant, and the method further comprises detecting motion of the human infant over time, mapping motion of the human infant over time to generate a baseline, and utilizing human infant motion trends to determine whether the human infant is undergoing proper development.

In certain embodiments, the method further comprises displaying, on an electronic display device, one of the following items relating to the mammalian subject: respiration rate, respiration history, alarm state, alarm history, non-respiratory motion history, and baseline values indicative of a pattern of normal movement.

In certain embodiments, the method further comprises communicating signals indicative of one or more of the following items over a communication network to a remotely located signal receiving device: respiration rate, respiration history, alarm state, alarm history, non-respiratory motion history, and baseline values indicative of a pattern of normal movement.

In certain embodiments, the method further comprises capturing one or more still images or videos of the mammalian subject, and storing the one or more still images or videos of the mammalian subject.

In certain embodiments, when an alarm signal is detected, the method further comprises storing one or more still images or videos of the mammalian subject in association with the processed signal values generated by or derived from the at least one processor indicative of at least one of (i) respiratory motion of the mammalian subject or (ii) non-respiratory motion of the mammalian subject.

In certain embodiments, the method further comprises capturing sounds generated by the mammalian subject, and storing the one or more sounds generated by the mammalian subject.

In certain embodiments, when an alarm signal is detected, the method further comprises storing one or more sounds generated by the mammalian subject in association with the processed signal values generated by or derived from the at least one processor indicative of at least one of (i) respiratory motion of the mammalian subject or (ii) non-respiratory motion of the mammalian subject.

In certain embodiments, any two or more aspects or embodiments or other features disclosed herein may be combined for additional advantage.

### BRIEF DESCRIPTION OF DRAWINGS

In certain embodiments, any two or more aspects or embodiments or other features disclosed herein may be combined for additional advantage.

FIG. 1 is a schematic diagram illustrating connections between various components of a system for remotely sensing periodic and non-periodic motion (e.g., respiration and non-respiratory motion) of a mammalian subject.

FIG. 2 illustrates various radio frequency components according to one implementation of the system described in connection with FIG. 1.

FIG. 3 schematically illustrates system utilizing a transceiver remotely located relative to a mammalian subject, with a radio frequency signal generated by the transceiver being impinged on tissue of the mammalian subject.

FIG. 4 is a schematic diagram showing components of a radio frequency radar transceiver apparatus useable with embodiments of the present disclosure.

FIG. 5 is a schematic diagram showing the radio frequency transmit antenna, radio frequency receive antenna, and radar transceiver circuitry of FIG. 4 and a processor, all mounted on a circuit board and arranged to communicate with an electronic device.

FIG. 6 is a flowchart identifying steps of a method for detecting at least one health state or health condition of a mammalian subject according to one embodiment of the present disclosure.

FIG. 7 is a schematic diagram of a generalized representation of a computer system that can be included as a component of the systems or methods disclosed herein.

### DETAILED DESCRIPTION

The present disclosure relates in various aspects to devices and methods utilizing reflectometric detection of a mammalian subject in order to detect health states or health conditions that may be correlated to respiration and/or motion (e.g., including compliance with or deviation from patterns thereof). Such detection is contactless in nature, without requiring physical contact with a subject. In one aspect, the disclosure relates to a system for remotely sensing movement of a mammalian subject. In another aspect, the disclosure relates to a method for detecting at least one health state or health condition of a mammalian subject.

Embodiments herein employ RF signals reflected from a mammalian body to detect respiration and motion detection to enable analysis of health conditions (e.g., respiratory-related conditions).

Doppler radar utilizes the theory that a reflected radar wave from a moving target will directly affect the frequency of the return signal. A radar wave reflected from a target moving in a periodic forward/backward motion will exhibit what can be classified as a phase shift relative to the periodic motion. A mammalian body exhibits this periodic motion when the mammal engages in respiration. If the body reflecting the RF wave also exhibits non-respiratory motion, then the reflected signal will also contain another component indicated by the type and magnitude of motion. The phase shift of the received signal can be analyzed for respiration and motion contents while filtering other noise components from the scene. If the motion originates from the movement of the subject under test in the same frequency band as the respiration, then the motion signal dominates the return signal and can mask the respiration signal itself.

Systems and methods for remotely sensing physiologic (e.g., cardiac) data of subjects have been disclosed in U.S. Pat. Nos. 9,492,099; 7,811,234; and 7,272,431. U.S. Pat. No. 7,811,234 discloses a non-imaging method of remotely sensing cardiac-related data of a subject, the method including: transmitting a microwave signal to illuminate tissue of the subject; receiving a reflected microwave signal, the reflected microwave signal being a reflection of the microwave signal from illuminated tissue of the subject; processing the reflected microwave signal and analyzing an amplitude of the reflected microwave signal to determine changes in a reflection coefficient at an air-tissue interface of the subject's body resulting from changes in permittivity of the illuminated tissue of the subject, the changes in permittivity containing a static component and a time-varying component; and processing the time-varying component to provide cardiographic related data of the subject. U.S. Pat. No. 9,492,099 discloses systems and methods for remote sensing of physiologic activity, including cardiac activity and respiration rate, with signal processing schemes to provide improved reproducibility despite variation in relative position between RF components and a human subject, movement of a human subject, and/or presence of interfering signals. In certain embodiments, hardware and/or filtering schemes of U.S. Pat. No. 9,492,099 may be used in implementations of systems and methods disclosed herein.

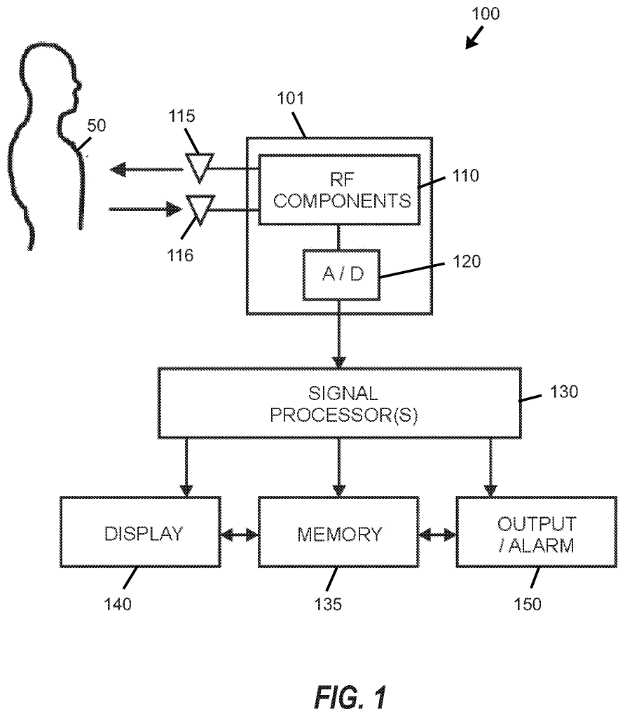

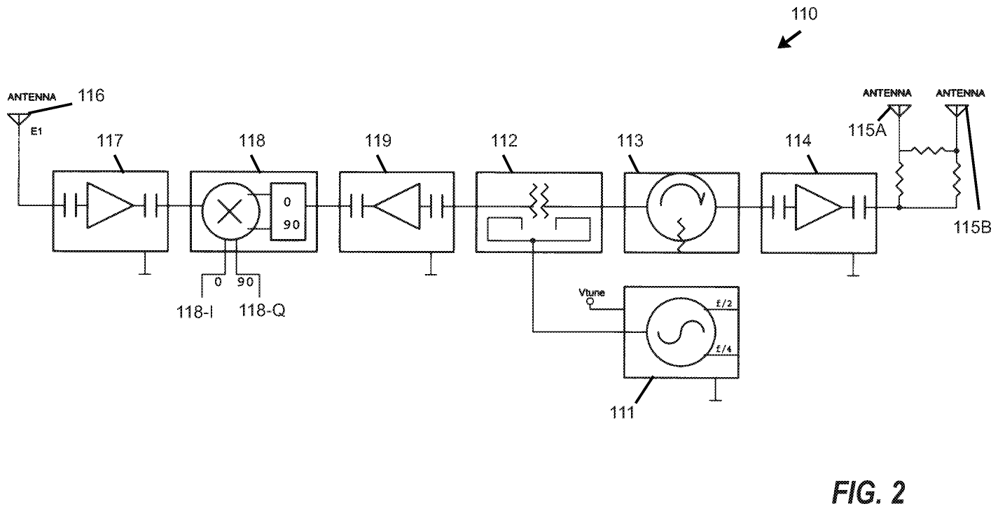

FIG. 1 illustrates connections between various components of a system 100 for remotely sensing periodic and non-periodic motion (e.g., respiration and non-respiratory motion) of a mammalian subject 50. At least one RF transmitter 115 and at least one RF receiver 116 are arranged in sufficient proximity to the mammalian subject 50 to enable a RF signal from the RF transmitter 115 to impinge on tissue of the mammalian subject 50, and to permit a reflection of the transmitted RF signal to be received by the RF receiver 116. Multiple RF transmitters and/or RF receivers may be used, such as may be useful to mitigate motion artifacts and/or detect multiple subjects in a sensing area. Although the RF transmitter 115 and RF receiver 116 are illustrated as being spatially separated, such components may be grouped or otherwise packaged in a single component (e.g., transceiver) or assembly. The RF transmitter 115 and RF receiver 116 are arranged in communication with RF components 110 (as described in further detail in FIG. 2) to facilitate transmission and detection of RF signals. A RF signal generated by the RF transmitter 115 may include a continuous wave signal, and is preferably a microwave signal (e.g., preferably in an unregulated RF band as 900 MHz, 2.4 GHz, 5.8 GHz, 10 GHz, 24 GHz, 60 GHz, or 77 GHz). The invention is not limited to use of continuous wave signals, since pulsed signals and/or other signals used in conventional radar (including Doppler radar) systems may be used, as will be apparent to one skilled in the art. An analog signal received from the RF receiver 116 is preferably converted to a baseband signal via the RF components 110 and then converted to a digital signal via at least one analog-to-digital converter 120. The RF components 110 and analog-to-digital converter 120 may be arranged on or in a single substrate and/or enclosure 101. Although preferred embodiments include use of at least one analog-to-digital converter 120, it is to be appreciated that the invention is not so limited, since one skilled in the art would appreciate that analog signals may be used and processed according to various methods disclosed herein without requiring digital conversion.

One or more signal processing components 130 are arranged to receive signals from the RF components 110 or signals derived therefrom. If signals generated by the RF components are not subject to analog-to-digital conversion, then the signal processing component(s) may include elements suitable for analog signal manipulation, such as capacitors, resistors, inductors, and transistors. In embodiments where signals from the RF components 110 are subjected to analog-to-digital conversion, the signal processing components 130 preferably embody at least one digital signal processor (processing component), such as a general purpose or special purpose microprocessor. Various functions that may be performed by one or more digital signal processors include filtering, zero-crossing detection, auto-correlation, periodicity determination, and rate computation. At least one memory element 135 is preferably arranged in communication with the one or more signal processing components 130. Additionally, at least one output and/or alarm element 150, and/or a display 140, may be arranged in communication with at least one of the signal processing components 130 and/or the memory element(s) 135. Any of various components or systems (not shown) may be connected to the output/alarm element 150, such as a control system, a communications interface, and/or other functional components.

FIG. 2 illustrates various RF components 110 according to one implementation of the system 100 described in connection with FIG. 1. An oscillator 111 is arranged to generate an oscillating wave signal at a desired frequency (e.g., 10 GHz, 24 GHz, 60 GHz, or 77 GHz). A splitter 112 divides the oscillating wave signal for use by the transmitting and receiving components. A circulator 113 is arranged to promote one-way flow (e.g., to the right) of a first split component of the oscillating wave signal toward a RF transmission signal amplifier 114 while attenuating any signals (e.g., noise) traveling in the opposing direction (e.g., to the left, toward the splitter 112). An amplified oscillating wave signal generated by the amplifier 114 is provided to one or more multiple RF transmitting antennas 115A, 115B, of a type (e.g., microwave) appropriate to the frequency generated by the oscillator 111.

A RF receiving antenna 116 is arranged to receive a reflected RF signal that includes a reflection of the RF signal transmitted by the transmitting antennas 115A, 115B and reflected from tissue of a mammalian subject. The RF signal received by the receiving antenna 116 is amplified by an amplifier 117 and then supplied to a quadrature mixer 118 that serves to mix at least a portion of a “transmitted” RF signal with the amplified received RF signal. The quadrature mixer 118 receives a split portion of the oscillating wave signal following passage through the splitter 112 and amplification by another amplifier 119. In one embodiment, the reflected RF signal comprises a real signal component (I) and an out-of-phase signal component (Q), wherein the quadrature mixer 118 is arranged to generate a baseband signal (or baseband data) that includes the real signal component (I) (via output line 118-1) and the out-of-phase signal component (Q) (via output line 118-Q). In another embodiment (according to an operating mode termed QLOCK™, which is a trademark of PROBE Science, Inc., Pasadena, Calif.), the out-of-phase signal component (Q) may be kept constant (e.g., by feeding voltage from an out of phase component (Q) back to a tuned voltage of the frequency channel (e.g., via input “Vtune” associated with the oscillator 111)), and in such embodiment the quadrature mixer 118 may be arranged to output a baseband signal including only the real signal component (I). In certain embodiments, the RF components may be arranged to transmit an encoded signal to permit selective identification at the receiving end of signals received from the transmitter, thereby facilitating identification and removal of interfering signals. Encoded signal transmission may be used in conjunction with either continuous wave or pulsed signal embodiments.

With the preceding introduction to reflectometric detection being completed, aspects and embodiments of the present disclosure will now be described in further detail.

Certain embodiments disclosed herein utilize an RF transceiver (or separate transmitter and receiver), antenna(s), an analog front end, an analog/digital converter, and a processor to transmit a RF signal and receive a reflected RF signal (i.e., reflected by a human being) in order to detect the presence of a human being, utilizing the Doppler radar principle. A combination of a hardware circuit and a software algorithm may be used to extract features from the reflected RF signal in order to separately detect respiratory motion and non-respiratory motion via signal processing of the reflected RF signal. Processed signal values generated by or derived from the at least one processor may be stored in a memory, the processed signal values being indicative of at least one of (i) respiratory motion of the mammalian subject or (ii) non-respiratory motion of the mammalian subject. Additionally, one or more processed signals generated by or derived from the at least one processor may be compared against one or more stored processed signal values (e.g., one or more values or patterns indicative of baseline or normal conditions), and responsive to the comparing, detecting at least one health state or health condition of the mammalian subject. Various corrective actions may be taken in response to such detection, such as summoning human and/or medical assistance for the mammalian subject, generating or more alarms, etc.

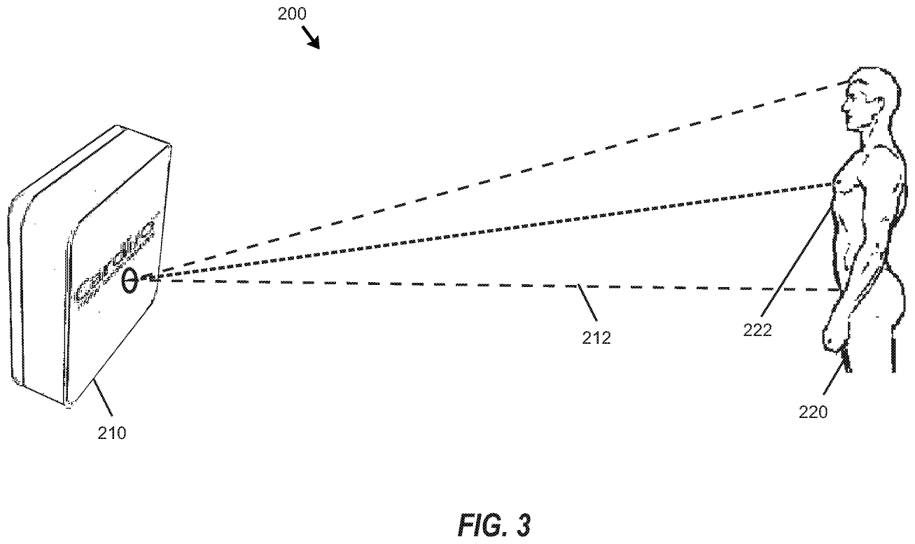

FIG. 3 schematically illustrates portions of a system 200 utilizing a transceiver 210 remotely located relative to a mammalian subject 220, wherein a RF signal 212 generated by the transceiver 210 is impinged on tissue 222 (including torso tissue) of the mammalian subject 220. The transceiver 210 may be located at any desirable distance from the mammalian subject 220, such as in a range of 6 inches to 20 feet or more; or in a range of 1-10 feet, or in a range of 1-5 feet, etc.

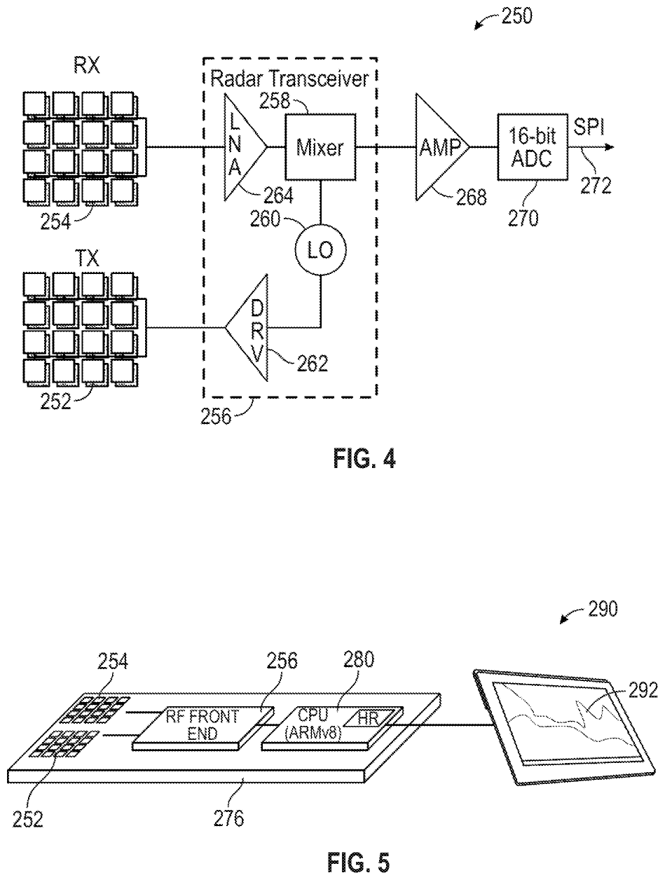

FIG. 4 is a schematic showing components of a RF radar transceiver apparatus useable with embodiments of the present disclosure. The RF radar transceiver apparatus 250 includes a RF transmit antenna 252 and a RF receive antenna 254 coupled with radar transceiver circuitry 256. The radar transceiver circuitry 256 includes a mixer 258, a local oscillator 260, and a RF antenna driver 262 configured to drive the transmit antenna 252. The radar transceiver circuitry 256 further includes a low noise amplifier 264 configured to receive signals from the RF receive antenna 254 and pass such signals to a mixer 258 that is coupled with the local oscillator 260. Signals are thereafter amplified by a second amplifier 268 and digitally converted by an analog to digital converter 270 and sent via a serial peripheral interface (SPI) 272 to at least one downstream processor for processing as disclosed herein.

In certain embodiments, the RF transmit antenna 252 may include multiple antennas and/or the RF receive antenna 254 may include multiple antennas, providing a phased array. In certain embodiments, phase may be dynamically adjusted between multiple antennas to cause a focus of the antennas to sweep across a field of view (in a manner similar to MIMO technology for WiFi/LTE transmission). Such scheme may enable more gain and a narrower field of view, with the narrow field of view enabling reduction of antenna size and/or tracking of multiple individuals in a given field of view.

FIG. 5 is a schematic showing the RF transmit antenna 252, RF receive antenna 254, and radar transceiver circuitry of FIG. 4 together with a processor 280 (e.g., ARM v8 CPU) all mounted on a circuit board 276 and arranged to communicate with an electronic device 290 which may include a display 292.

In certain embodiments, a directional transmit patch antenna may be fabricated as copper on a controlled impedance (e.g., Rogers) printed circuit board. The patch antenna may be designed for a particular field of view that is appropriate to cover the area (width and distance) of interest in front of the display unit, and will vary based upon the configuration of an electronic device incorporating the display. The receive antenna may be a separate instance of the same or different field of view in the case of a continuous wave (CW) RF signal. According to certain embodiments utilizing pulsed radar, a single antenna can act as the transmit and receive antenna. The transmit antenna may be fed via 50-ohm matched transmission lines to an RF transceiver. On the transmit side, the transceiver may generate a frequency specific carrier at the designed frequency operational point from a local oscillator. This frequency may be static in the case of CW, or may be chirped in the case of FM-CW. This transmit signal may be passed through the transmit antenna, and propagates through free space and any obstructions until it hits a surface that reflects the signal. The reflected signal will be modulated by the motion of the object. Free space path loss and other obstructions will attenuate the signal as a function of the square of the distance in each direction. A small portion of the reflected signal further reduced by the radar cross section of the subject, hits the effective aperture of the RF receive antenna and will pass through a 50-ohm matched transmission line to the receive input of the RF transceiver.

The transceiver may heterodyne the received signal with the local oscillator, and output the baseband in phase and quadrature components of the received signal. This signal contains the modulation present as a result of the properties of the reflected object in addition to a phase shift based on distance, and a coherent and non-coherent phase noise component.

In certain embodiments, quadrature outputs may be sent to an analog front for amplification. The reflected baseband signal may be severely attenuated due to free space path loss, LCD screen materials, and low radar cross section of a human body. Such signal may be too low to be detected natively. The analog amplification may be accomplished utilizing ultra-low noise amplifiers. An analog gain of between 10× and 1000× is used to cover the ranges of interest for a presence detect system. The signal is then passed to an n-bit ADC (where n is at least 16) and sampled at a rate of at least 50 sps. The ADC stores the n-bit samples of the in-phase and quadrature components. A processor with supporting firmware reads these samples from the registers of the ADC, when the ADC signals data is available via its interrupt signal. The data samples may then passed onto an algorithm engine which processes the data for presence information.

Once in the algorithm engine on a processor (e.g., programmable processor or fixed function ASIC), the data samples may be vector processed for their content. The post processed samples are filtered to remove out of band noise, phase noise components as well as compensate for the phase delay based on the distance to the target object. The samples are then passed to an algorithm that detects gross motion. Motion is determined by fast large variations in the signal amplitude indicated by derivatives of the post processed signal.

Motion is tracked and baselined per subject via an artificial intelligence (AI) engine. The AI engine may also be trained with what one or more expert practitioners may determine as normal motion patterns. Deviations from either of these can be logged and alarmed as needing further attention. The indication of motion is then fed to the respiration extraction algorithm.

The respiration extraction algorithm processes the signal in the time domain by band passing and down sampling the data into the frequencies of interest for respiration. It then performs complex demodulation on the I and Q channels and determines the channel that is providing the highest quality signal. The signal is then smoothed to remove artifacts and then frequency analyzed for content. There will be one, two or more indications of frequency content in the signal. The respiration would be the dominate except in the case of motion. The motion detection algorithm feeds forward into the decision of the frequency domain analysis. By providing an indication of the quantity and type of motion as indicated by its algorithm as described earlier, the respiration engine can identify the frequency component related to the motion versus the one that is indicative of the respiration. The respiration algorithm then reports and respiration rate which can then be smoothed or averaged and presented to a user.

In certain embodiments, data samples are analyzed to extract the phase modulation that corresponds to respiration-related movement of the reflected body, and separately extract movement of the reflected body not corresponding to respiration-related movement. The algorithm can determine the respiration rate in breaths per minute by analyzing the phase shift versus time.

In certain embodiments, apnea may be detected by the fact that the diaphragm of the subject will cease movement either with or without motion. Apnea without motion is detected via an algorithm that operates in the time domain, and detects when the signal level phase shift has ceased or slowed to the point where it crosses a threshold. When the signal crosses this threshold, it has been determined that the mammalian subject has ceased respiration functions.

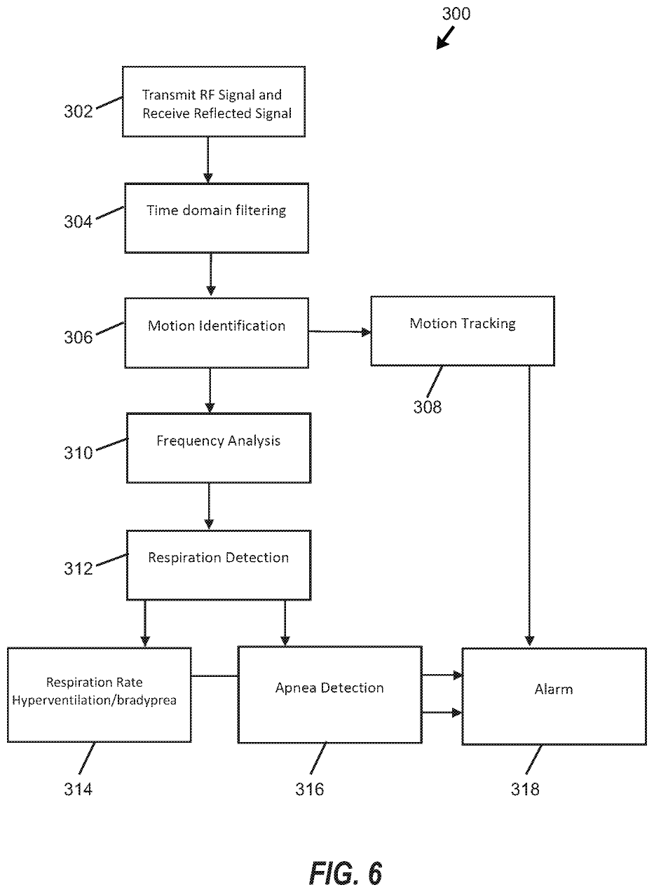

FIG. 6 is a flowchart identifying steps in a method for detecting at least one health state or health condition of a mammalian subject. Starting at block 302, RF signal is transmitted to impinge on tissue of a mammalian subject, preferably including a torso thereof, and RF signal comprising a reflection of the RF signal impinged on tissue of a mammalian subject is received. Continuing to block 304, time domain filtering is performed on the received RF signal. Continuing to block 306, motion identification is performed using the filtered RF signal. Frequency analysis is performed thereafter, according to block 310, with such analysis permitting detection of respiratory-related motion according to block 312. Respiration detection permits identification of respiration rate, thereby permitting identification of (i) hyperventilation or bradypnea states according to block 314 or (ii) an apnea state, according to block 316. Turning back to the motion identification block 306, non-respiratory motion may be detected and tracked according to block 308. Results of the non-respiratory motion tracking 308 as well as respiratory motion tracking 312 may be utilized in combination to detecting at least one health state or health condition of a mammalian subject, and to generate one or more local and/or remote alarms (optionally utilizing wired or wireless network communication) according to block 318.

In certain embodiments, a transmitted RF signal resides in the microwave frequency band.

In certain embodiments, a reflected RF signal is analyzed and extracted to identify at least one non-respiratory motion pattern.

In certain embodiments, a reflected RF signal is analyzed and extracted for a respiration rate.

In certain embodiments, a respiration detection algorithm is notified by a motion detection algorithm of the presence and characteristics of the motion.

In certain embodiments, a respiration detection algorithm can identify the frequency components of the respiration separately from those of non-respirator motion of the mammalian subject.

In certain embodiments, motion activity is tracked, baselined, and passed through an AI algorithm for anomaly detection.

In certain embodiments, detection of the absence of a respiration signal indicates an apnea event.

In certain embodiments, detection of unusually high respiration rates indicated a hyperventilation event.

In certain embodiments, detection of unusually low respiration rates indicates a bradypnea event.

In certain embodiments, an anomalous event related to respiratory motion and/or non-respiratory motion (either separately or combined) can be alarmed to provide indication of necessary intervention.

In certain embodiments, systems and methods herein may report a respiration rate via the signal processing of the received RF signal when the subject is in the field of view of the transmitting RF antenna.

In certain embodiments, sensitivity of the receiver may be dynamically adjusted to account for variations in the placement of the RF sensor relative to the mammalian subject being monitored or under test.

In certain embodiments, alarm states may be generated upon detection of conditions such as apnea, hyperventilation, bradypnea, lack of respiration, or the like.

In certain embodiments, motion of a subject may be mapped over time in order to baseline and compare to motion trends indicating proper development of a human infant. In certain embodiments, motion deviations from an AI trained baseline for a human infant may trigger notification or alarm.

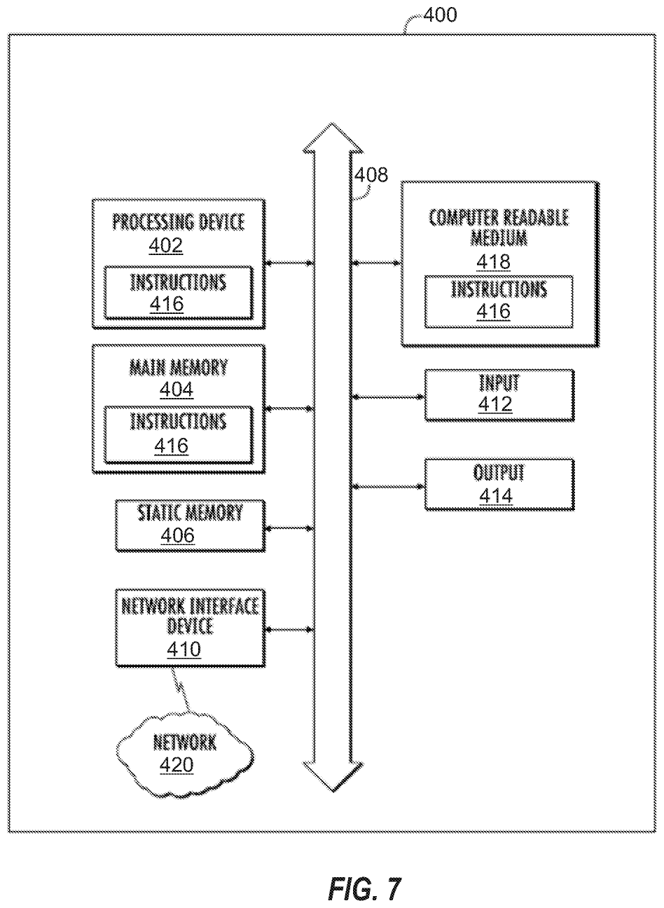

FIG. 7 is a schematic diagram of a generalized representation of a computer system 400 (optionally embodied in a computing device) that can be included in any component of the systems or methods disclosed herein. In this regard, the computer system 400 is adapted to execute instructions from a computer-readable medium to perform these and/or any of the functions or processing described herein. The computer system 400 in FIG. 7 may include a set of instructions that may be executed to program and configure programmable digital signal processing circuits for supporting scaling of supported communication services. The computer system 400 may be connected (e.g., networked) to other machines in a LAN, an intranet, an extranet, or the Internet. While only a single device is illustrated, the term “device” shall also be taken to include any collection of devices that individually or jointly execute a set (or multiple sets) of instructions to perform any one or more of the methodologies discussed herein. The computer system 400 may be a circuit or circuits included in an electronic board card, such as a printed circuit board (PCB), a server, a personal computer, a desktop computer, a laptop computer, a personal digital assistant (PDA), a computing pad, a mobile device, or any other device, and may represent, for example, a server or a user's computer.

The computer system 400 in this embodiment includes a processing device or processor 402, a main memory 404 (e.g., read-only memory (ROM), flash memory, dynamic random access memory (DRAM), such as synchronous DRAM (SDRAM), etc.), and a static memory 406 (e.g., flash memory, static random access memory (SRAM), etc.), which may communicate with each other via a data bus 408. Alternatively, the processing device 402 may be connected to the main memory 404 and/or static memory 406 directly or via some other connectivity means. The processing device 402 may be a controller, and the main memory 404 or static memory 406 may be any type of memory.

The processing device 402 represents one or more general-purpose processing devices, such as a microprocessor, central processing unit, or the like. More particularly, the processing device 402 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 402 is configured to execute processing logic in instructions for performing the operations and steps discussed herein.

The computer system 400 may further include a network interface device 410. The computer system 400 also may or may not include an input 412, configured to receive input and selections to be communicated to the computer system 400 when executing instructions. The computer system 400 also may or may not include an output 414, including but not limited to a display, a video display unit (e.g., a liquid crystal display (LCD) or a cathode ray tube (CRT)), an alphanumeric input device (e.g., a keyboard), and/or a cursor control device (e.g., a mouse).

The computer system 400 may or may not include a data storage device that includes instructions 416 stored in a computer readable medium 418. The instructions 416 may also reside, completely or at least partially, within the main memory 404 and/or within the processing device 402 during execution thereof by the computer system 400, the main memory 404 and the processing device 402 also constituting computer readable medium. The instructions 416 may further be transmitted or received over a network 420 via the network interface device 410.

While the computer readable medium 418 is shown in an embodiment to be a single medium, the term “computer-readable medium” should be taken to include a single medium or multiple media (e.g., a centralized or distributed database, and/or associated caches and servers) that store the one or more sets of instructions. The term “computer readable medium” shall also be taken to include any medium that is capable of storing, encoding, or carrying a set of instructions for execution by the processing device 402 and that cause the processing device 402 to perform any one or more of the methodologies of the embodiments disclosed herein. The term “computer readable medium” shall accordingly be taken to include, but not be limited to, solid-state memories, optical media, and magnetic media.

The embodiments disclosed herein include various steps. The steps of the embodiments disclosed herein may be executed or performed by hardware components or may be embodied in machine-executable instructions, which may be used to cause a general-purpose or special-purpose processor programmed with the instructions to perform the steps. Alternatively, the steps may be performed by a combination of hardware and software.

The embodiments disclosed herein may be provided as a computer program product, or software, that may include a machine-readable medium (or computer readable medium) having stored thereon instructions which may be used to program a computer system (or other electronic devices) to perform a process according to the embodiments disclosed herein. A machine-readable medium includes any mechanism for storing or transmitting information in a form readable by a machine (e.g., a computer). For example, a machine-readable medium includes: a machine-readable storage medium (e.g., ROM, random access memory (“RAM”), a magnetic disk storage medium, an optical storage medium, flash memory devices, etc.); and the like.

Unless specifically stated otherwise and as apparent from the previous discussion, it is appreciated that throughout the description, discussions utilizing terms such as “analyzing,” “processing,” “computing,” “determining,” “displaying,” or the like, refer to the action and processes of a computer system, or a similar electronic computing device, that manipulates and transforms data and memories represented as physical (electronic) quantities within registers of the computer system into other data similarly represented as physical quantities within the computer system memories or registers or other such information storage, transmission, or display devices.

The algorithms and displays presented herein are not inherently related to any particular computer or other apparatus. Various systems may be used with programs in accordance with the teachings herein, or it may prove convenient to construct more specialized apparatuses to perform the required method steps. The required structure for a variety of these systems is disclosed in the description above. In addition, the embodiments described herein are not described with reference to any particular programming language. It will be appreciated that a variety of programming languages may be used to implement the teachings of the embodiments as described herein.

Those of skill in the art will further appreciate that the various illustrative logical blocks, modules, circuits, and algorithms described in connection with the embodiments disclosed herein may be implemented as electronic hardware, instructions stored in memory or in another computer readable medium and executed by a processor or other processing device, or combinations of both. The components of the system described herein may be employed in any circuit, hardware component, integrated circuit (IC), or IC chip, as examples. Memory disclosed herein may be any type and size of memory and may be configured to store any type of information desired. To clearly illustrate this interchangeability, various illustrative components, blocks, modules, circuits, and steps have been described above generally in terms of their functionality. How such functionality is implemented depends on the particular application, design choices, and/or design constraints imposed on the overall system. Skilled artisans may implement the described functionality in varying ways for each particular application, but such implementation decisions should not be interpreted as causing a departure from the scope of the present embodiments.

The various illustrative logical blocks, modules, and circuits described in connection with the embodiments disclosed herein may be implemented or performed with a processor, a Digital Signal Processor (DSP), an Application Specific Integrated Circuit (ASIC), a Field Programmable Gate Array (FPGA), or other programmable logic device, a discrete gate or transistor logic, discrete hardware components, or any combination thereof designed to perform the functions described herein. Furthermore, a controller may be a processor. A processor may be a microprocessor, but in the alternative, the processor may be any conventional processor, controller, microcontroller, or state machine. A processor may also be implemented as a combination of computing devices (e.g., a combination of a DSP and a microprocessor, a plurality of microprocessors, one or more microprocessors in conjunction with a DSP core, or any other such configuration).

The embodiments disclosed herein may be embodied in hardware and in instructions that are stored in hardware, and may reside, for example, in RAM, flash memory, ROM, Electrically Programmable ROM (EPROM), Electrically Erasable Programmable ROM (EEPROM), registers, a hard disk, a removable disk, a CD-ROM, or any other form of computer readable medium known in the art. A storage medium is coupled to the processor such that the processor can read information from, and write information to, the storage medium. In the alternative, the storage medium may be integral to the processor. The processor and the storage medium may reside in an ASIC. The ASIC may reside in a remote station. In the alternative, the processor and the storage medium may reside as discrete components in a remote station, base station, or server.

It is also noted that the operational steps described in any of the embodiments herein are described to provide examples and discussion. The operations described may be performed in numerous different sequences other than the illustrated sequences. Furthermore, operations described in a single operational step may actually be performed in a number of different steps. Additionally, one or more operational steps discussed in the embodiments may be combined. Those of skill in the art will also understand that information and signals may be represented using any of a variety of technologies and techniques. For example, data, instructions, commands, information, signals, bits, symbols, and chips, which may be referenced throughout the above description, may be represented by voltages, currents, electromagnetic waves, magnetic fields, particles, optical fields, or any combination thereof.

Unless otherwise expressly stated, it is in no way intended that any method set forth herein be construed as requiring that its steps be performed in a specific order. Accordingly, where a method claim does not actually recite an order to be followed by its steps, or it is not otherwise specifically stated in the claims or descriptions that the steps are to be limited to a specific order, it is in no way intended that any particular order be inferred.

While the invention has been has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Various combinations and sub-combinations of the structures described herein are contemplated and will be apparent to a skilled person having knowledge of this disclosure. Any of the various features and elements as disclosed herein may be combined with one or more other disclosed features and elements unless indicated to the contrary herein. Correspondingly, the invention as hereinafter claimed is intended to be broadly construed and interpreted, as including all such variations, modifications and alternative embodiments, within its scope and including equivalents of the claims.

What is claimed is:

1. A system for remotely sensing movement of a mammalian subject, the system comprising:
a radio frequency (RF) transmitter configured to transmit a RF signal for impingement on tissue of the mammalian subject;
a RF receiver configured to receive a RF signal comprising a reflection of the RF signal impinged on tissue of the mammalian subject;
at least one processor configured to process the received RF signal to separately identify presence of respiratory motion of the mammalian subject and presence of non-respiratory motion of the mammalian subject; and
a memory configured to store processed signal values generated by or derived from the at least one processor indicative of at least one of (i) respiratory motion of the mammalian subject or (ii) non-respiratory motion of the mammalian subject;
wherein the at least one processor is further configured to compare one or more processed signals generated by or derived from the at least one processor against one or more stored processed signal values, and detect at least one health state or health condition of the mammalian subject.

2. The system of claim 1, wherein the at least one processor is additionally configured to initiate at least one of the following actions (i) or (ii) responsive to detection of at least one health state or health condition correlated to deviation from baseline conditions for respiratory and/or non-respiratory movement of the mammalian subject: (i) generate an alarm signal, or (ii) summon human and/or medical assistance for the mammalian subject.

3. The system of claim 1, wherein the at least one health state or health condition of the mammalian subject comprises a respiratory trauma event, an apnea event, a bradypnea event, a hyperventilation event, a choking event, or a suffocating event experienced by the mammalian subject.

4. The system of claim 1, wherein the one or more processed signals generated by or derived from the at least one processor comprise one or more baseline values indicative of at least one pattern of normal movement of the mammalian subject, and detection of the at least one health state or health condition of the mammalian subject comprises deviation from the at least one pattern of normal movement of the mammalian subject.

5. The system of claim 4, wherein the at least one processor is configured to generate the one or more baseline values using an artificial intelligence engine.

6. The system of claim 1, wherein the mammalian subject comprises a human infant or child, and the RF transmitter comprises a direction RF transmitter that is mounted and aligned to point at a torso of the human infant or child when the human infant or child is present in a medical apparatus, a crib, a bed, or an infant safety seat.

7. The system of claim 1, wherein the mammalian subject comprises a human infant, and the system is configured to detect motion of the human infant over time, to map motion of the human infant over time to generate a baseline, and to utilize the human infant motion trends to determine whether the human infant is undergoing proper development.

8. The system of claim 1, further comprising a camera configured to image the mammalian subject, wherein the memory is configured to store one or more still images or videos of the mammalian subject.

9. The system of claim 8, wherein when an alarm signal is detected, the memory is configured to store one or more still images or videos of the mammalian subject in association with the processed signal values generated by or derived from the at least one processor indicative of at least one of (i) respiratory motion of the mammalian subject or (ii) non-respiratory motion of the mammalian subject.

10. The system of claim 1, further comprising a microphone configured to capture sounds generated by the mammalian subject, wherein the memory is configured to store one or more sounds generated by the mammalian subject.

11. A method for detecting at least one health state or health condition of a mammalian subject, the method comprising:
transmitting a radio frequency (RF) signal to impinge on tissue of the mammalian subject;
receiving a RF signal comprising a reflection of the RF signal impinged on tissue of the mammalian subject;
processing the received RF signal utilizing at least one processor to separately identify presence of respiratory motion of the mammalian subject and presence of non-respiratory motion of the mammalian subject;
storing processed signal values generated by or derived from the at least one processor in a memory, the processed signal values being indicative of at least one of (i) respiratory motion of the mammalian subject or (ii) non-respiratory motion of the mammalian subject; and
utilizing the at least one processor, comparing one or more processed signals generated by or derived from the at least one processor against one or more stored processed signal values, and responsive to the comparing, detecting at least one health state or health condition of the mammalian subject.

12. The method of claim 11, further comprising automatically taking at least one of the following actions (i) or (ii) responsive to detection of at least one health state or health condition correlated to deviation from baseline conditions for respiratory and/or non-respiratory movement of the mammalian subject: (i) generating an alarm signal, or (ii) summoning human and/or medical assistance for the mammalian subject.

13. The method of claim 11, wherein the at least one health state or health condition of the mammalian subject comprises a respiratory trauma event, an apnea event, a bradypnea event, a hyperventilation event, a choking event, or a suffocating event experienced by the mammalian subject.

14. The method of claim 11, wherein the processed signals generated by or derived from the at least one processor comprise one or more baseline values indicative of at least one pattern of normal movement of the mammalian subject, and the detection of the at least one health state or health condition of the mammalian subject comprises identification of deviation from the at least one pattern of normal movement of the mammalian subject.

15. The method of claim 14, further comprising generating the one or more baseline values using an artificial intelligence engine.

16. The method of claim 11, wherein the mammalian subject comprises a human infant or child, and the RF transmitter is mounted and aligned to point at a torso of the human infant or child when the human infant or child is present in a medical apparatus, a crib, a bed, or an infant safety seat.

17. The method of claim 11, wherein the mammalian subject comprises a human infant, and the method further comprises detecting motion of the human infant over time, mapping motion of the human infant over time to generate a baseline, and utilizing the human infant motion trends to determine whether the human infant is undergoing proper development.

18. The method of claim 11, further comprising communicating signals indicative of one or more of the following items over a communication network to a remotely located signal receiving device: respiration rate, respiration history, alarm state, alarm history, non-respiratory motion history, and baseline values indicative of a pattern of normal movement.

19. The method of claim 11, further comprising capturing one or more still images of the mammalian subject, videos of the mammalian subject, or sounds generated by the mammalian subject, and storing the one or more still images, videos, or sounds.

20. The method of claim 19, wherein when an alarm signal is detected, the method further comprises storing one or more still images of the mammalian subject, videos of the mammalian subject, or sounds generated by the mammalian subject in association with the processed signal values generated by or derived from the at least one processor indicative of at least one of (i) respiratory motion of the mammalian subject or (ii) non-respiratory motion of the mammalian subject.
